(19) United States
(12) Patent Application Publication
(10) Pub. No.: US 2019/0254740 A1
(43) Pub. Date: Aug. 22, 2019

(54) DEVICES, SYSTEMS, AND METHODS FOR REGULATING GLUCOSE LEVELS INCLUDING TREATING DIABETES

(71) Applicant: Boston Scientific Scimed, Inc., Maple Grove, MN (US)

(72) Inventors: Vijay Koya, Blaine, MN (US); Elizabeth M. Annoni, White Bear Lake, MN (US); Bryan A. Clark, Forest Lake, MN (US); Bruce Forsyth, Hanover, MN (US); Hong Cao, Maple Grove, MN (US); Matthew R. DeWitt, Charlottesville, VA (US); Kyle H. Srivastava, Saint Paul, MN (US)

(21) Appl. No.: 16/279,438

(22) Filed: Feb. 19, 2019

Related U.S. Application Data
(60) Provisional application No. 62/632,811, filed on Feb. 20, 2018.

(51) Int. Cl. A61B 18/14; A61M 37/00; G16H 20/17

(52) U.S. Cl. CPC A61B 18/1492; A61B 2018/00875; G16H 20/17; A61M 37/0015

(57) ABSTRACT

Devices, systems, and methods for regulating glucose levels, including treating diabetes, in accordance with the present disclosure may include a catheter having an expandable or inflatable portion, one or more electrodes disposed on the expandable or inflatable portion of the catheter, wherein the electrodes are configured to deliver energy to a patient's gastrointestinal tract, and a drug delivery mechanism for delivering a drug therapy subsequent to energy delivery by the electrodes. A method for regulating glucose levels according to the present disclosure may include inserting a catheter into a patient's gastrointestinal tract, positioning the catheter in a duodenum of the patient's gastrointestinal tract, expanding or inflating a portion of the catheter in the duodenum, the expandable or inflatable portion of the catheter including electrodes, applying energy to the duodenum via the electrodes to ablate tissue of the duodenum, and delivering a drug therapy to the ablated tissue of the duodenum.

### CROSS-REFERENCE TO RELATED APPLICATIONS

This application is a non-provisional application of, and claims the benefit of priority to, U.S. Provisional Application Ser. No. 62/632,811, filed Feb. 20, 2018, entitled “Devices, Systems, and Methods for Regulating Glucose Levels Including Treating Diabetes,” the entirety of which application is expressly incorporated by reference herein.

### FIELD

The present disclosure relates generally to devices, systems, and methods for regulating glucose levels and, more particularly, to electroporation devices, systems, and methods for treating diabetes.

### BACKGROUND

Diabetes is a disease affecting a significant proportion of the population resulting in substantial medical costs worldwide. Existing pharmacological treatments may not be sufficient in regulating glucose levels in patients, and may also cause side effects such as hypoglycemia, gastrointestinal (GI) complications, peripheral edema, body weight increases, and the like. Consequently, patients may not adhere to proper drug treatment over time. Patients may therefore not be able to achieve desired glycemic levels, thereby potentially increasing medical costs over their lifetime and increasing risk of medical complications.

It is with respect to these and other considerations that the present improvements may be useful.

### SUMMARY

This Summary is provided to introduce a selection of concepts in a simplified form that are further described below in the Detailed Description. This Summary is not intended to necessarily identify key features or essential features of the claimed subject matter, nor is it intended as an aid in determining the scope of the claimed subject matter.

According to an exemplary embodiment of the present disclosure, a system for regulating glucose levels may include a catheter having an expandable or inflatable portion, one or more electrodes disposed on the expandable or inflatable portion of the catheter, wherein the one or more electrodes are configured to deliver energy to a patient's gastrointestinal tract, and a drug delivery mechanism for delivering a drug therapy subsequent to energy delivery by the one or more electrodes.

In various of the foregoing and other embodiments of the present disclosure, the drug delivery mechanism may include a drug-coated balloon, one or more microneedles, an implantable device, or a hydrogel, or combinations thereof. Energy may be deliverable by electroporation. The drug therapy may include a growth inhibitor, cell cycle regulatory proteins/molecules, cyclin-dependent kinases, cell cycle inhibitors, cell regeneration inhibitor agents, or simulants of growth inhibitors, or combinations thereof. Growth inhibitors may include extracellular proteins, growth receptors, growth factors, transcriptional factors, cell adhesion molecules, cell signaling molecules, cytokines and chemokines, sulfate proteoglycans, chondroitin sulfate proteoglycans, enzymes, arginase, 13-secretase, or urokinase-type and tissue-type plasminogen activators, or combinations thereof. Extracellular proteins may include laminin, fibronectin, tenascin, fibrinogen, or fibrin, or combinations thereof. The growth inhibitors may include growth receptors including tyrosine kinase receptors (e.g., TrkA, TrkB, and/or TrkC), common neurotrophic receptor (e.g., P75NTR), ErbB receptors, or fibroblast growth factor receptors, or combinations thereof. The growth factors may include transforming growth factor alpha (TGF-α), epidermal growth factor (EGF), transforming growth factor beta (TGF-β), insulin-like growth factor (IGF), colony-stimulating factor (CSF), fibroblast growth factor (FGF), trefoil factor (TFF), hepatocyte growth factor (HGF), Glucagon-like peptide (GLP-2), or growth hormone (GH), or combinations thereof. The transcriptional factors may include the Hedgehog family, Forkhead Box (FOX) factors, Homeobox (HOX) genes, ParaHox genes, GATA transcription factors, canonical Wnt/β-catenin signaling, EPH/Ephrins, BMP signaling, K-RAS, Notch pathway, or HNF or MATH1, or combinations thereof. The cell adhesion molecules (CAM) may include N-CAM, Ng-CAM/L1, or N-cadherin or L2-HWk-1, or combinations thereof. The cell signaling molecules may include Ras, Phosphotidyl Inositol 3-kinase, Phospholipase c-gamma 1, mitogen activated phosphor kinase, protein kinase A, Jaks/STATs signaling molecules, or combinations thereof. The kinase inhibitors may include staurosporine, H 89, dihydrochloride, cAMPS-Rp, triethylammonium salt, KT 5720, wortmannin, LY294002, 1C486068, 187114, GDC-0941, Gefitinib, Erlotinib, Lapatinib, AZ623, K252a, KT-5555, Cyclotraxin-B, Lestaurtinib, Tofacitinib, Ruxolitinib, SB1518, CYT387, LY3009104, TG101348, WP-1034, PD173074, or SPRY4, or combinations thereof. The cytokines and chemokines may include transforming growth factor-α, epidermal growth factor, interleukin-1β, or interferon-γ, or combinations thereof. The sulfate proteoglycans may include keratin sulfate proteoglycans, and wherein the chondroitin sulfate proteoglycans may include neurocan, brevican, versican, phosphacan, aggrecan, or NG2, or combinations thereof. The enzymes may include targeting enzymes including Arginase I, Chondroitinase ABC, 13-secretase BACE1, urokinase-type plasminogen activator, or tissue-type plasminogen activator, or combinations thereof. Arginase I may include an N-hydroxy-L-arginine or 2(S)-amino-6-boronohexonic acid, or combinations thereof. 13-secretase may include N-Benzyloxycarbonyl-Val-Leu-leucinal, H-Glu-Val-Asn-Statine-Val-Ala-Glu-Phe-NH2, or H-Lys-Thr-Glu-Glu-Ile-Ser-Glu-Val-Asn-Stat-Val-Ala-Glu-Phe-OH, or combinations thereof. The urokinase-type and tissue-type plasminogen activators may include serpin E1, Tiplaxtinin, or plasminogen activator inhibitor-2, or combinations thereof. The cell cycle regulatory proteins/molecules may include Cyclin A, Cyclin D, Cyclin D, Cyclin E, or Cyclin B, or combinations thereof. The cyclin-dependent kinases may include Cdk1, Cdk2, Cdk3, Cdk4, or Cdk6, or combinations thereof. The cell cycle inhibitors may include p21, p27, or p57, or combinations thereof. The cell regeneration inhibitor agents may include paclitaxel, dual phosphate and tensin homolog (PTEN), or SCOS3, or combinations thereof. Simulants of growth inhibitors may include INK4a/ARF families including p16 and p14. The one or more electrodes may be configured to deliver an amount of energy sufficient to ablate tissue of the patient's gastrointestinal tract, and the drug therapy may be deliverable in an amount sufficient to inhibit subsequent cell growth of the tissue.

According to an exemplary embodiment of the present disclosure, a method for regulating glucose levels may include inserting a catheter into a patient's gastrointestinal tract, positioning the catheter in a duodenum of the patient's gastrointestinal tract, and expanding or inflating a portion of the catheter in the duodenum. The expandable or inflatable portion of the catheter may include one or more electrodes. The method may further include applying energy to the duodenum via the one or more electrodes to ablate tissue of the duodenum. The method may further include delivering a drug therapy to the ablated tissue of the duodenum. The drug therapy may include a growth inhibitor, cell cycle regulatory proteins/molecules, cyclin-dependent kinases, cell cycle inhibitors, cell regeneration inhibitor agents, or simulants of growth inhibitors, or combinations thereof, including the examples provided above.

According to an exemplary embodiment of the present disclosure, a method of treating diabetes may include applying electroporation energy to a duodenum of a patient to ablate tissue of the duodenum, and delivering a drug therapy to the ablated tissue of the duodenum. The drug therapy may include a growth inhibitor, cell cycle regulatory proteins/molecules, cyclin-dependent kinases, cell cycle inhibitors, cell regeneration inhibitor agents, or simulants of growth inhibitors, or combinations thereof, including the examples provided above.

In various of the foregoing and other embodiments of the present disclosure, drug therapy may be delivered by a drug-coated balloon, one or more microneedles, an implantable device, or a hydrogel, or combinations thereof. The application of the energy and delivery of the drug therapy may alter how the patient's body regulates glucose levels. The drug therapy may include a growth inhibitor, cell cycle regulatory proteins/molecules, cyclin-dependent kinases, cell cycle inhibitors, cell regeneration inhibitor agents, or simulants of growth inhibitors, or combinations thereof, including the examples provided above.

### BRIEF DESCRIPTION OF THE DRAWINGS

Non-limiting embodiments of the present disclosure are described by way of example with reference to the accompanying figures, which are schematic and not intended to be drawn to scale. In the figures, each identical or nearly identical component illustrated is typically represented by a single numeral. For purposes of clarity, not every component is labeled in every figure, nor is every component of each embodiment shown where illustration is not necessary to allow those of ordinary skill in the art to understand the disclosure. In the figures:

FIGS. 1A, 1B and 2 illustrate gastrointestinal anatomy of a human patient;

FIG. 3 illustrates an exemplary embodiment of an energy delivery device of a system in accordance with the present disclosure;

FIG. 4 illustrates another exemplary embodiment of an energy delivery device of a system in accordance with the present disclosure;

FIG. 5 illustrates another exemplary embodiment of an energy delivery device of a system in accordance with the present disclosure;

FIG. 6 illustrates another exemplary embodiment of a drug therapy delivery device of a system in accordance with the present disclosure;

FIG. 7 illustrates another exemplary embodiment of a system in accordance with the present disclosure;

FIG. 8 illustrates another exemplary embodiment of a system in accordance with the present disclosure;

FIG. 9 illustrates an exemplary embodiment of an electroporation delivery system and components in accordance with the present disclosure;

FIG. 10 illustrates an exemplary embodiment of a processing device of an electroporation delivery system in accordance with the present disclosure;

FIG. 11 illustrates an exemplary embodiment of a storage medium of an electroporation delivery system in accordance with the present disclosure; and

FIG. 12 illustrates an exemplary embodiment of a computing architecture of an electroporation delivery system in accordance with the present disclosure.

### DETAILED DESCRIPTION

The present disclosure is not limited to the particular embodiments described herein. The terminology used herein is for the purpose of describing particular embodiments only, and is not intended to be limiting beyond the scope of the appended claims. Unless otherwise defined, all technical terms used herein have the same meaning as commonly understood by one of ordinary skill in the art to which the disclosure belongs.

As used herein, the singular forms “a,” “an,” and “the” are intended to include the plural forms as well, unless the context clearly indicates otherwise. It will be further understood that the terms “comprises” and/or “comprising,” or “includes” and/or “including” when used herein, specify the presence of stated features, regions, steps elements and/or components, but do not preclude the presence or addition of one or more other features, regions, integers, steps, operations, elements, components and/or groups thereof.

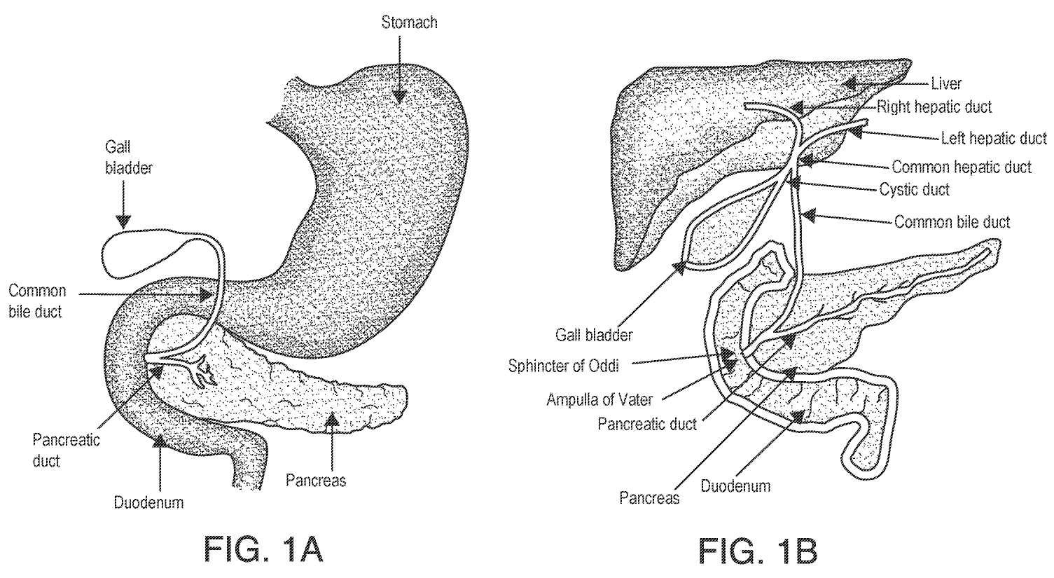

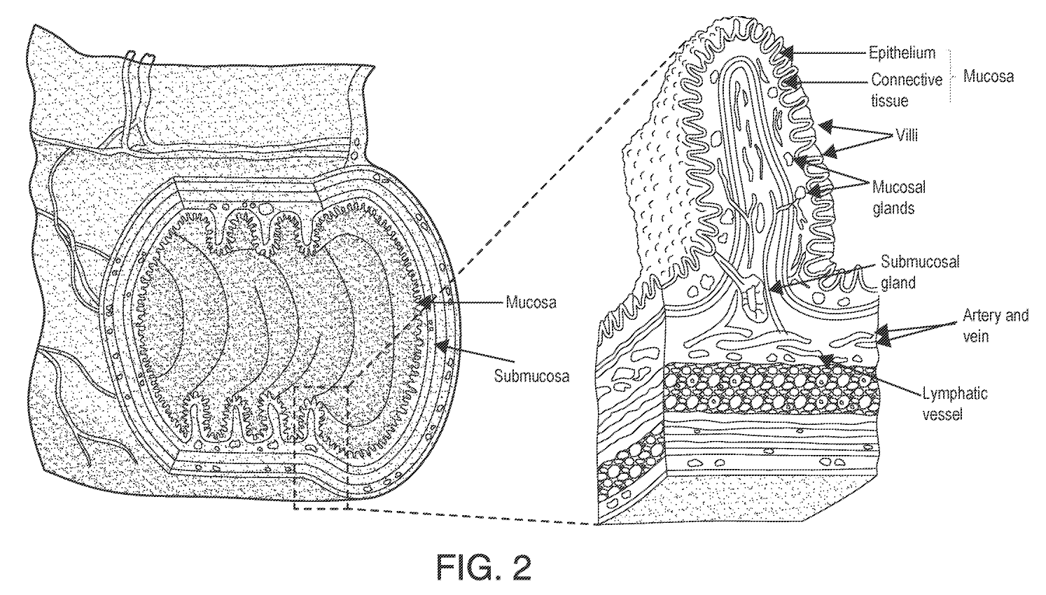

As described above, pharmacological treatment alone may be insufficient for treating diabetes in a patient over their lifetime. In accordance with exemplary embodiments of the present disclosure, devices, systems, and methods for regulating blood glucose levels (e.g., treating diabetes) may more effectively regulate glycemic levels, e.g., via neuromodulation of hepatic nerves. For example, the duodenal mucosa of a patient's gastrointestinal tract (see FIGS. 1A, 1B, and 2) may be manipulated by an athermal modality, e.g., electroporation, to ablate the mucosal epithelium. After an ablation procedure, a drug may be administered to slow and/or prevent regrowth of the mucosal epithelium. This may modulate or regulate the glycemic control in a safer and more sustainable manner.

As shown in FIG. 2, a mucosal wall of the duodenum has a plurality of folds, e.g., accordion or bellow-shaped. The mucosa includes an epithelium layer that may be targeted for treatment in accordance with the present disclosure. For example, ablation procedures may cause cell death in the epithelium layer, and drug therapies may be applied before, simultaneously, and/or subsequent to the ablation procedure to slow and/or eliminate regrowth. In embodiments where a drug therapy is delivered subsequent to an electroporation procedure, the drug therapy may be deliverable via a same or a separate (e.g., independently-delivered catheter) expandable device from the expandable device delivering the electroporation therapy. In embodiments where a drug therapy is delivered prior and/or simultaneously to an electroporation procedure, the drug therapy may be deliverable via the same expandable device as delivering the electroporation therapy.

In some embodiments, electroporation may be deliverable endoscopically, for example, by one or more electrodes disposed on a balloon or a balloon catheter, without causing significant thermal heating or collateral cell death through thermal necrosis. In this manner, diabetes, in particular, type-2 diabetes, may be treatable by perhaps altering the surface of the duodenal mucosa to alter downstream signaling and eliciting metabolic improvement. Other mechanisms of action elicited by application of ablative energy followed by drug therapy may be possible with similar effect. It is understood that in other embodiments, other modalities may be used to deliver energy, including but not limited to electrical energy, microwave energy, ultrasound energy, radiofrequency energy, focused ultrasound (e.g., high-intensity focused ultrasound (HIFU) and/or low-intensity focused ultrasound (LIFU)), laser energy, infrared energy, light energy, thermal energy steam or heated water, magnetic fields, reversible electroporation, cryogenic therapy, brachytherapy, ionizing therapy, drug delivery, biologic delivery, chemical ablation (e.g., ethanol), mechanical disruption, or any other therapy modality to alter or otherwise cause disruption or modulation of the target tissue, or combinations thereof. Additionally, methods of treating tissue, such as described in U.S. patent application Ser. No. 14/833,585, entitled “Devices for Damaging Nerves and Related Methods of Use,” which is herein incorporated by reference in its entirety, may also find application in the present disclosure.

Electroporation, e.g., irreversible electroporation (IRE), may be advantageous over other ablation therapies as it is an athermal process, so as to minimize or eliminate a need for insulation during the procedure, thereby reducing risk to the patient. For example, patients undergoing treatments may be at risk for potential inflammatory responses and/or resulting strictures caused by thermal systems. IRE may also be beneficial in that IRE treatment may not affect surrounding tissue architecture collateral to the target area, including but not limited to collagen, arteries, veins, ducts, nerves, and vasculature. Thus, IRE may be deliverable near vital organs without risk or without significant risk of causing damage that may otherwise occur through conventional thermal ablation techniques.

A procedure including energy delivery (e.g., IRE) and application of a drug therapy may avoid risks of thermal systems. In some embodiments, treatment zones may be utilized, e.g., one or more non-fully circumferential treatment zones to further reduce patient risk. For example, a treatment zone of any circumferential area less than 360° (e.g., approximately 90°, 180°, 270°, or any other angle) may provide a targeted area of treatment to reduce risk to a patient. Treatment zones may be used in addition to an IRE procedure, and/or may be used in other energy delivery modalities as described above. In some embodiments, a treatment zone may be determined by identifying abnormal tissue (e.g., a diseased portion of tissue). The treatment zone may only target diseased tissue to reduce a potential negative reaction (e.g., inflammatory response) and thereby lower a patient's risk of post-operative complications. A sensor, e.g., an optical sensor, may be included in a treatment system, using for example hyperspectral and/or multispectral imaging. The sensor may identify desired locations for a therapy to be delivered.

In an embodiment of a treatment procedure, electrical impedance may be used to differentiate between healthy mucosal tissue and pathological tissue, e.g., within the small intestine. In diabetes, structural deviations may occur in the small intestine including but not limited to surface area, elevated number of goblet cells per villus, decreased muscle thickness with connective tissue infiltration, reduced number of Auerbach's plexuses, lymphocyte aggregations accompanied by blunted villi, blood vascular lesions, and/or deformed villi due to excessive loss of epithelial cells, or combinations thereof. These morphological differences may contribute to the differences in electrical impedance between healthy and pathological tissue. These differences in electrical impedances may then be used to guide the medical professional to ablate pathological tissue while sparing healthy tissue.

IRE may apply monopolar, e.g., with a set of grounding pads, bipolar, and/or multipolar high-voltage electrical pulses to achieve desired cell death. In some embodiments, electrical pulses may be delivered between approximately 1000V and 5000V, and may be deliverable in pulses in a range from approximately 0.1 to 100 μsec. Electrical pulses may be biphasic, or dual polarity. Delivery of electrical pulses may be tunable to reduce patient muscle contraction. In some embodiments, a series of pulses, e.g., approximately 1 to 100 μsec may be deliverable, and can also be synchronizable with a patient's heart rhythms. As such, the treatment procedure may be completed in a very short time, e.g., less than 5 minutes, or approximately 1 to 3 minutes.

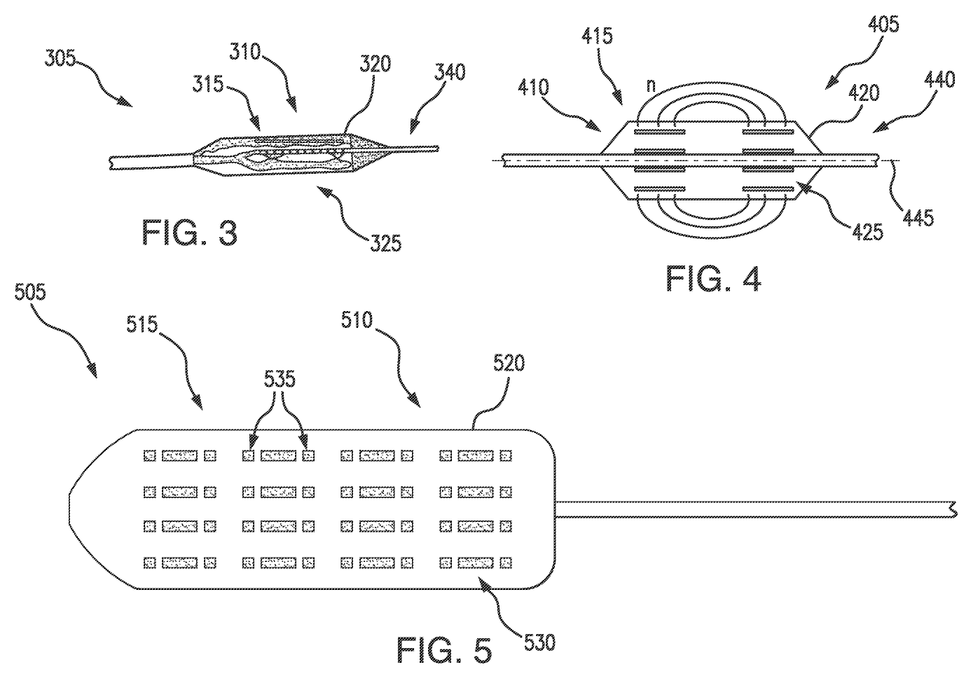

In some embodiments, a device for administering electroporation may be deliverable to a patient's duodenum by a catheter and expandable for a treatment procedure. An endoscope (not shown) may deliver a catheter and additional tools and devices for treatment. For example, an expandable balloon or balloon catheter may include one or more electrodes for administering electroporation. Referring now to FIGS. 3-5, exemplary embodiments of an energy delivery device in a system for treating diabetes in accordance with the present disclosure are shown. A device 305, 405, 505 may include a respective catheter 315, 415, 515, e.g., a balloon catheter 315, 415, 515, which may be at least partially expandable and/or inflatable once positioned in the patient's duodenum. For example, a balloon may be expandable along at least a portion of the catheter shaft, thereby providing tissue apposition along the whole or a portion of a length of the balloon. The balloon may be deflated, repositioned, and re-inflated to provide a longer region of treatment. In some embodiments, the expandable and/or inflatable portion 310, 410, 510 of the respective catheter 315, 415, 515 may be a balloon. For example, the catheter 315, 415, 515 may be at least partially self-expanding upon exiting a sheath or expandable by a medical professional after correct placement is verified. The catheter 315, 415, 515 may be at least partially expandable to any size so as to fill the duodenum lumen of the patient It is also understood that the catheter 315, 415, 515 may be non-balloon based. For example, instead of an expandable balloon, the catheter 315, 415, 515 may be at least partially formed of a shape memory material and/or include mechanically-activated splines, e.g., as an expandable scaffold, cage or splines with free terminal ends.

As shown in FIGS. 1A-1B, the duodenum may have a curvature, e.g., a C-shape, and may be approximately 35-38 cm in length. A catheter 315, 415, 515 may be flexible and/or adaptable to conform to the patient's duodenum. For example, the expandable and/or inflatable portion 310, 410, 510 of the catheter 315, 415, 515, e.g., the balloon, may be formed of a PET material, or may be a Nybex balloon. In some embodiments, the balloon portion of the catheter 315, 415, 515 may be approximately 6 mm×20 mm.

In some embodiments, the catheter 315, 415 may include a tip 340, 440. The tip 340, 440 may be an atraumatic, soft deflectable tip to reduce risk of perforation as the catheter 315, 415, 515 is advanced in the duodenum of the patient.

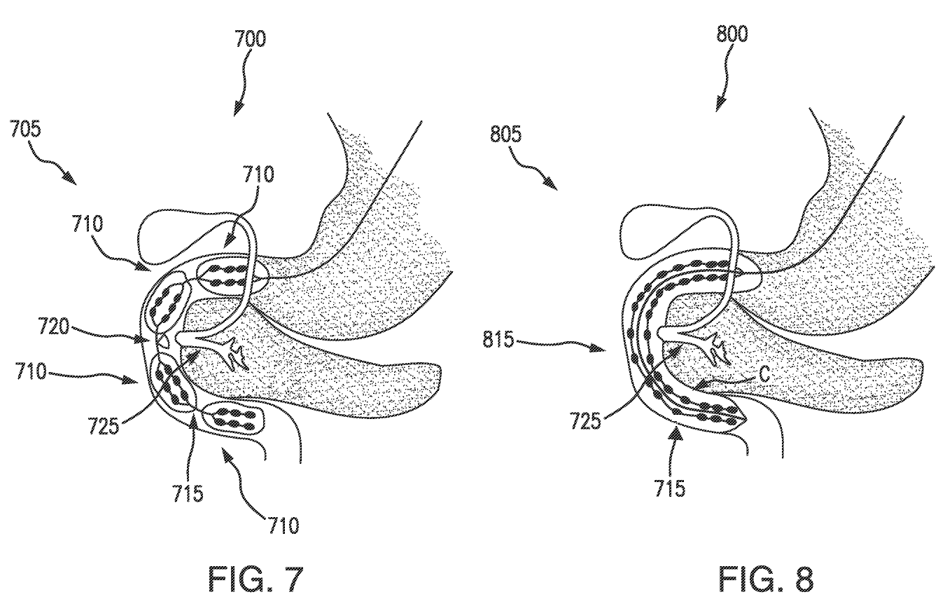

Although FIGS. 3-5 illustrate a single balloon, in some embodiments, the device 305, 405, 505 may include a plurality of expandable/inflatable portions, e.g., balloons, so as to optimize navigation around curved areas of the duodenum. For example, multiple balloons may prevent straightening of soft tissue of the duodenum, and may allow treatment over a longer segment of the duodenum. Referring now to FIG. 7, an exemplary embodiment of a system 700 in accordance with the present disclosure is shown. A catheter 705 may include a plurality of expandable or inflatable portions, e.g., balloons, 710. As such, the portions 710 may follow the curvature of the duodenum 715.

In some embodiments, the catheter may be curved so that when the balloon is expanded, the balloon may have a curvature to substantially align with the curvature of a patient's duodenum. Referring now to FIG. 8, another exemplary embodiment of a system 800 in accordance with the present disclosure is shown. A catheter 805 may include a single balloon 815 having a curvature C. In embodiments, the curvature C may follow the duodenum, to optimize delivering energy to ablate the mucosa.

In accordance with the exemplary embodiments illustrated in FIGS. 3-5, 7, and 8, a catheter 315, 415, 515, 705, 805 may be insertable in a patient's gastrointestinal tract, and positionable at the duodenum. At least a portion of the respective catheter 315, 415, 515, 705, 805 may be expandable or inflatable to fill the duodenum such that an exterior surface 320, 420, 520 of the expandable or inflatable portion is adjacent to the mucosa wall of the duodenum.

In some embodiments, the catheter 315, 415, 515 may include one or more electrodes 325, 425, 530, 535 disposed on a respective exterior surface 320, 420, 520 of the balloon catheter 315, 415, 515. In some embodiments, pairs of electrodes 325, 425, 530, 535 e.g., approximately 3 to 5 pairs, may be disposed on the respective surface 320, 420, 520 of the balloon catheter 315, 415, 515. The electrodes 325, 425, 530, 535 may be flexible, and in some embodiments made from a polyimide, or a similar material, and/or may be plated as a gold electrode with controlled length and distance.

The electrodes 325, 425, 530, 535 may be disposable on an expandable and/or inflatable portion 310, 410, 510 of the catheter 315, 415, 515 in various patterns, which may deliver electroporation to a patient in a desired manner. For example, as shown in FIG. 3, the electrodes 325 may be disposed in a circumferential pattern around the exterior surface 320 of the expandable portion of the catheter 315. FIG. 4 illustrates electrodes 425 disposed in a longitudinal pattern (e.g., electrodes 425 disposed in a direction along longitudinal axis 445) along the exterior surface 420 of the expandable portion of the catheter 415. Electrodes may also be disposed in a spiral, staggered, or other pattern resulting in a partially circumferential pattern at any one axial location. Avoiding a fully circumferential electrode pattern may further reduce risk of stenosis to patients. In some embodiments, electrode configuration may allow an energy delivery modality (e.g., IRE) to be tailored to precisely target the desired region of tissue in a patient's duodenum, thereby optimizing therapy and reducing unnecessary damage on an individual patient basis. Different electrode patterns, including but not limited to the positioning, sizing, and spacing, may affect at least the gradient and magnitude of the electrical field depending on the applied potential (voltage). Electrodes may be spaced on the expandable and/or inflatable portion 310, 410, 510 to prevent arc-over.

When the expandable and/or inflatable portion 310, 410, 510 of the catheter 315, 415, 515 is delivered to the desired position in the patient's gastrointestinal tract, and inflated to expand (see, e.g., FIGS. 7-8), the electrodes 325, 425, 530, 535 may directly contact tissue. For example, the electrodes 325, 425, 530, 535 may directly contact the mucosa. It may be advantageous for the electrodes 325, 425, 530, 535 to directly contact tissue when the expandable and/or inflatable portion 310, 410, 510 of the catheter 315, 415, 515 is inflated/expanded to enable sufficient electrical connectivity and propagation into the tissue wall and the mucosal surface. In embodiments, a respective anode and cathode of an electrode circuit may be at least 1 cm apart. In some embodiments, e.g., in a monopolar configuration, a ground electrode positioned on a patient's skin surface may be spaced apart from electrodes on the balloon surface.

In some embodiments, as illustrated in FIG. 5, the catheter 415 may include one or more electroporation electrodes 530 and impedance measurement electrodes 535. The impedance measurement electrodes 535 may measure an impedance of electroporation electrodes 530. Impedance measurement electrodes 535 may aid in mapping prior to electroporation delivery, e.g., to determine anatomical targets to treat and/or avoid. Mapping may occur after the catheter 515 is inflated or expanded, e.g., by conducting an impedance test between multiple pairs of electrodes to generate an impedance map. For example, regions of the Ampulla of Vater (see FIG. 1B) may be expected to show higher impedance based on various factors, including but not limited to the type of tissue as compared to the surrounding mucosal tissue, a presence of water or other conductive media, a difference in tissue thickness, and/or a presence of blood or other heat sink or conductive media, or combinations thereof. Mapping of the duodenal region may allow a medical professional to avoid treatment of the Ampulla of Vater, regions adjacent to the pancreas (e.g., to reduce risk of pancreatitis), or other regions. Additionally, post-ablation, e.g., after IRE is delivered to the patient's duodenum, impedance measurement electrodes 535 may provide measurements of lesion sizes and may provide confirmation to the medical professional which regions have been ablated.

The catheter 315, 415, 515 may further include one or more imaging devices and/or lighting devices for a medical professional to visually determine positioning of the catheter. In some embodiments, a camera may be disposed for radially imaging a patient's duodenum, which may allow a medical professional to more accurately position the catheter 315, 415, 515 relative to an area to be protected, such as the pancreatic duct. For example, a camera 720 may be alignable with the pancreatic duct 725, to ensure proper positioning of the balloons when expanded. In some embodiments, a radial camera 720 may be disposed between balloons 710 (see FIG. 7). An imaging device, e.g., an ultrasound transducer, and in particular, a radial ultrasound transducer, may image the duodenal wall when the balloon is expanded.

When the catheter 315, 415, 515 is properly positioned in the duodenum, energy may be delivered to ablate the mucosa. As described above, IRE may be used for therapy delivery. IRE may be synchronized to a patient's electrocardiogram (ECG), for therapy delivery during the absolute myocardial refractory period after the R-wave of a patient's heartbeat, to minimize risk of arrhythmias. This energy delivery may ablate, or cause cell death, of tissue of the duodenum. For example, the epithelium layer may be ablated.

Subsequent to, or during, or after, electroporation procedures or other energy delivery therapy, a drug therapy may be applied to the inner duodenal surface (e.g., the ablated tissue)14, which may inhibit or reduce glucose uptake by modifying cellular structures, glucose break down and absorption, and the like. For example, combination drug therapy may be applied to avoid, inhibit, or prevent functional mucosal regrowth, which may in turn inhibit or reduce glucose uptake by modifying cellular structure.

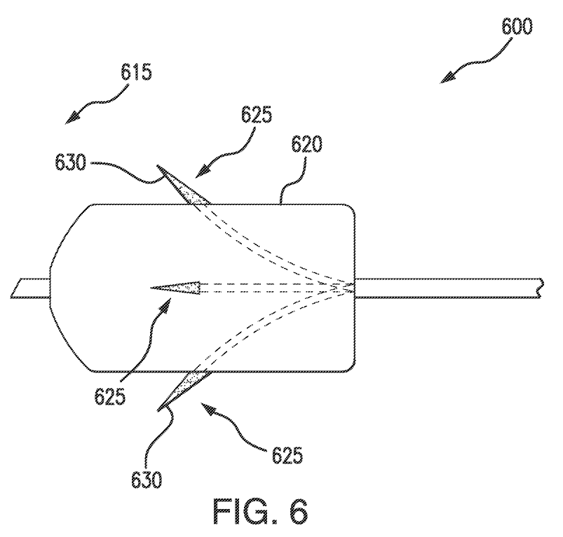

Drug therapies may be deliverable by various drug delivery mechanisms. Drug therapy delivered to a patient's duodenum subsequent to energy delivery, such as an electroporation procedure, may be advantageous in achieving sustained therapy to better regulate glucose levels through reduction and/or elimination of intestinal mucosal regeneration. For example, in some embodiments, drugs may be deliverable via one or more drug-coated balloons. In some embodiments, the drug therapy may be coated on the expandable/inflatable portion of the catheter 315, 415, 515. In other embodiments, needles, or microneedles may inject drug therapy into the duodenum. Referring now to FIG. 6, a device 600 may include a catheter 615, in which the catheter 615 is at least partially expandable and/or inflatable. The catheter 615 may be configured for needles (e.g., microneedles) 625 to extend beyond an outer surface 620 of the catheter 615. In some embodiments, drugs may be coated on tips 630 of the needles 625, so that after extending out of the outer surface 320 of the catheter 615, the tips 630 of the needles 625 may pierce the duodenal tissue, thereby depositing the drugs. In some embodiments, the needles 625 may be hollow, so that liquid drugs may be injectable into tissue. For example, when the tips 630 of the needles 625 pierce the duodenal tissue, the medical professional may inject a desired drug therapy into the duodenal tissue.

After the procedure, e.g., after a predetermined amount of time has elapsed (for example, approximately 10-20 days), the patient may be evaluated. For example, blood glucose levels may be evaluated via an oral glucose tolerance test, and/or hemoglobin A1c test. Results may be compared to blood glucose levels of the patient taken prior to the treatment. A decrease in the diagnostic values may be considered as a successful therapy to the patient. Blood glucose levels may be monitored over an extended period of time for longer-term evaluation.

In some embodiments, drug therapies may be deliverable via an implantable device. For example, the implantable device may be deliverable to the duodenum from a working channel of an endoscope. The implantable device may be a capsule, or other biodegradable device that may be placed in the duodenum lumen that releases a drug therapy over an extended period of time, e.g., in accordance with a predetermined release profile. In other embodiments, a hydrogel may be applied to the duodenum lumen, e.g., for sustained release. A hydrogel may be advantageous in maintaining a high localized concentration of a drug therapy over an extended period of time, and promote healthy cell regrowth. In embodiments, release mechanisms may include diffusion controlled, swelling controlled, chemically controlled, or environmentally-responsive release, or combinations thereof.

Drug therapies may include growth inhibitors, cell cycle regulatory proteins/molecules, cyclin-dependent kinases, cell cycle inhibitors, cell regeneration inhibitor agents, and/or simulants of growth inhibitors, or combinations thereof. Classes of growth inhibitors may target the cell division, replication, and/or regeneration machinery, and may advantageously slow and/or eliminate regrowth of ablated mucosa or other tissue.

Drug therapies may include individual or combination delivery of alpha-gluocosidase inhibitors, transport inhibitors (e.g., SLGT1 and/or GLUT2 inhibitors) and/or chemicals that induce fibrosis. Classes of growth inhibitors may be any of several categories, including but not limited to extracellular proteins, growth receptors, growth factors, transcriptional factors, cell adhesion molecules, cell signaling molecules, cytokines and chemokines, sulfate proteoglycans, chondroitin sulfate proteoglycans, enzymes, arginase, 13-secretase, and urokinase-type and/or tissue-type plasminogen activators. Classes may be determinable based on their function. A delivery method, e.g., described in accordance with the present disclosure, may be consistent across the classes. In some embodiments, other deliveries methods are envisioned.

Extracellular proteins may be utilized, such as laminin, fibronectin, tenascin, fibrinogen, and/or fibrin. Growth receptors may include tyrosine kinase receptors (e.g., TrkA, TrkB, and/or TrkC), common neurotrophic receptor (e.g., P75NTR), ErbB receptors, and/or fibroblast growth factor receptors. Growth factors may include transforming growth factor alpha (TGF-α), epidermal growth factor (EGF), transforming growth factor beta (TGF-β), insulin-like growth factor (IGF), colony-stimulating factor (CSF), fibroblast growth factor (FGF), trefoil factor (TFF), hepatocyte growth factor (HGF), Glucagon-like peptide (GLP-2), and/or growth hormone (GH). Transcriptional factors may be selected for regulating intestinal gene expression, including but not limited to the Hedgehog family, Forkhead Box (FOX) factors, Homeobox (HOX) genes, ParaHox genes, GATA transcription factors, canonical Wnt/β-catenin signaling, EPH/Ephrins, BMP signaling, K-RAS, Notch pathway, and/or HNF and MATH1. Cell adhesion molecules (CAM) may include N-CAM, Ng-CAM/L1, and/or N-cadherin and L2-HWk-1. Cell signaling molecules may include Ras, Phosphotidyl Inositol 3-kinase, Phospholipase c-gamma 1, mitogen activated phosphor kinase, protein kinase A, and/or Jaks/STATs signaling molecules. Additionally, cell signaling molecules may include kinase inhibitors including but not limited to staurosporine, H 89, dihydrochloride, cAMPS-Rp, triethylammonium salt, KT 5720, wortmannin, LY294002, 1C486068, 187114, GDC-0941, Gefitinib, Erlotinib, Lapatinib, AZ623, K252a, KT-5555, Cyclotraxin-B, Lestaurtinib, Tofacitinib, Ruxolitinib, SB1518, CYT387, LY3009104, TG101348, WP-1034, PD173074, and/or SPRY4. Cytokines and chemokines may include transforming growth factor-α, epidermal growth factor, interleukin-1β, and/or interferon-γ. Sulfate proteoglycans may include keratin sulfate proteoglycans. Chondroitin sulfate proteoglycans may include neurocan, brevican, versican, phosphacan, aggrecan, and/or NG2. Enzymes may include targeting enzymes including but not limited to Arginase I, Chondroitinase ABC, 13-secretase BACE1, urokinase-type plasminogen activator, and/or tissue-type plasminogen activator. Arginase I may include an N-hydroxy-L-arginine and/or 2(S)-amino-6-boronohexonic acid. 13-secretase may include N-Benzyloxycarbonyl-Val-Leu-leucinal, H-Glu-Val-Asn-Statine-Val-Ala-Glu-Phe-NH2, and/or H-Lys-Thr-Glu-Glu-Ile-Ser-Glu-Val-Asn-Stat-Val-Ala-Glu-Phe-OH. Urokinase-type and tissue-type plasminogen activators may include serpin E1, Tiplaxtinin, and/or plasminogen activator inhibitor-2.

In some embodiments, drug therapies may include cell cycle regulatory proteins/molecules, including but not limited to Cyclins, in particular Cyclin A, Cyclin D, Cyclin E, and/or Cyclin B. In some embodiments, cyclin-dependent kinases may include Cdk1, Cdk2, Cdk3, Cdk4, and/or Cdk6. In some embodiments, cell cycle inhibitors may be of the cip/kip family including p21, p27, and/or p57. Cell regeneration inhibitor agents may include Paclitaxel, dual phosphate and tensin homolog (PTEN), and/or SOCS3. PTEN may reduce regeneration at least partially by limiting mTOR activity and protein synthesis, and SOCS3 may inhibit regeneration by affecting gene transcription. In some embodiments, simulants of growth inhibitors may include INK4a/ARF family including p16 and/or p14. P16 may bind to CDK4 and arrests the cell cycle in G1 phase. P14 may prevent p53 degradation.

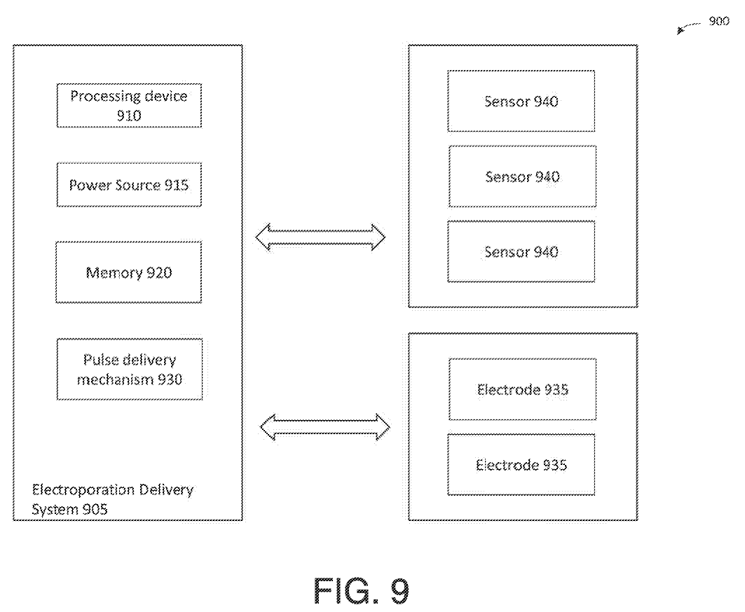

An electroporation delivery system, such as an IRE delivery system, of which an exemplary embodiment in accordance with the present disclosure is illustrated in the block diagram 900 of FIG. 9, may be used for energy delivery in the systems described herein. In some embodiments, an electroporation delivery system 905 may include several components for operation, including but not limited to a processing device 910, a power source 915, a memory 920, and a pulse delivery mechanism 930, which are described below. The electroporation delivery system 905 may be operatively connected to one or more electrodes 935 for delivering pulses by the pulse delivery mechanism 930 in an electroporation treatment in a patient's gastrointestinal tract. The electrodes may be configured for delivery to the gastrointestinal tract and application of an electroporation pulse, with delivery platforms, e.g., a catheter having an expandable or inflatable portion, for delivery of electroporation energy.

In the following description, numerous specific details such as processor and system configurations are set forth in order to provide a more thorough understanding of the described embodiments. However, the described embodiments may be practiced without such specific details. Additionally, some well-known structures, circuits, and the like have not been shown in detail, to avoid unnecessarily obscuring the described embodiments.

One or more flow charts for carrying out the executed steps/methods of the disclosure may be provided. Although such figures presented herein may include a particular process flow, it can be appreciated that the flow charts merely provide an example of how the general functionality as described herein can be implemented. Further, the given flow charts do not necessarily have to be executed in the order presented unless otherwise indicated. In addition, the given processes may be implemented by a hardware element, a software element executed by a processor, or any combination thereof. For example, the processes may be implemented by a processor component executing instructions stored on an article of manufacture, such as a storage medium. A storage medium may comprise any non-transitory computer-readable medium or machine-readable medium, such as an optical, magnetic or semiconductor storage. The storage medium may store various types of computer executable instructions, such as instructions to implement one or more disclosed processes. Examples of a computer readable or machine readable storage medium may include any tangible media capable of storing electronic data, including volatile memory or non-volatile memory, removable or non-removable memory, erasable or non-erasable memory, writeable or re-writeable memory, and so forth. Examples of computer executable instructions may include any suitable type of code, such as source code, compiled code, interpreted code, executable code, static code, dynamic code, object-oriented code, visual code, and the like. The embodiments are not limited in this context.

Referring back to FIG. 9, the electroporation delivery system 905 may execute processing operations or logic for the monitoring of the patient and electroporation pulse delivery using the processing device 910. The processing device 910 may comprise various hardware elements, software elements, or a combination of both. Examples of hardware elements may include devices, logic devices, components, processors, microprocessors, circuits, processor circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), memory units, logic gates, registers, semiconductor device, chips, microchips, chip sets, and so forth. Examples of software elements may include software components, programs, applications, computer programs, application programs, system programs, software development programs, machine programs, operating system software, middleware, firmware, software modules, routines, subroutines, functions, methods, procedures, software interfaces, application program interfaces (API), instruction sets, computing code, computer code, code segments, computer code segments, words, values, symbols, or any combination thereof. Determining whether an embodiment is implemented using hardware elements and/or software elements may vary in accordance with any number of factors, such as desired computational rate, power levels, heat tolerances, processing cycle budget, input data rates, output data rates, memory resources, data bus speeds and other design or performance constraints, as desired for a given implementation.

In some embodiments, the electroporation delivery system 905 may execute communications operations or determination of delivery an electroporation pulse using a communications component (not shown). The communications component may implement any well-known communications techniques and protocols, such as techniques suitable for use with packet-switched networks (e.g., public networks such as the Internet, private networks such as an enterprise intranet, and so forth), circuit-switched networks (e.g., the public switched telephone network), or a combination of packet-switched networks and circuit-switched networks (with suitable gateways and translators). The communications component may include various types of standard communication elements, such as one or more communications interfaces, network interfaces, network interface cards (NIC), radios, wireless transmitters/receivers (transceivers), wired and/or wireless communication media, physical connectors, and so forth. By way of example, and not limitation, communication media may include wired communications media and wireless communications media. Examples of wired communications media may include a wire, cable, metal leads, printed circuit boards (PCB), backplanes, switch fabrics, semiconductor material, twisted-pair wire, co-axial cable, fiber optics, a propagated signal, and so forth. Examples of wireless communications media may include acoustic, radio-frequency (RF) spectrum, infrared and other wireless media.

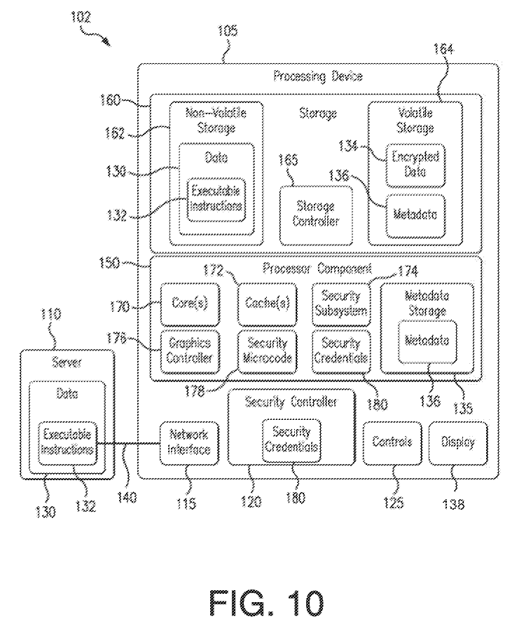

Turning now to FIG. 10, illustrated is an example of an operating environment, which may be used monitor and determine when to deliver electroporation pulses to a patient, a system 102 may include a server 110 and a processing device 105, which may be the same or similar to the electroporation delivery system 905 of FIG. 9, coupled via a network 140. Server 110 and processing device 105 may exchange data 130 via network 140, and data 130 may include executable instructions 132 for execution within processing device 105. In some embodiments, data 130 may be include data values, executable instructions, and/or a combination thereof. In other embodiments, data 130 may include sensor metric data from the sensors 940 and electrode data from the electrodes 935 of FIG. 9. Network 140 may be based on any of a variety (or combination) of communications technologies by which signals may be exchanged, including without limitation, wired technologies employing electrically and/or optically conductive cabling, and wireless technologies employing infrared, radio frequency, and/or other forms of wireless transmission.

In various embodiments, processing device 105 may incorporate a processor component 150, which may be the same or similar to the processing device 910 of FIG. 9, a storage 160, controls 125 (for instance, manually-operable controls), a display 138 and/or a network interface 115 to couple the processing device 105 to the network 140. Processor component 150 may incorporate security credentials 180, a security microcode 178, metadata storage 135 storing metadata 136, a security subsystem 174, one or more processor cores 170, one or more caches 172 and/or a graphics controller 176. Storage 160 may include volatile storage 164, non-volatile storage 162, and/or one or more storage controllers 165. Processing device 105 may include a controller 120 (for example, a security controller) that may include security credentials 180. Controller 120 may also include one or more of the embodiments described herein for unified hardware acceleration of hash functions.

Volatile storage 164 may include one or more storage devices that are volatile in as much as they require the continuous provision of electric power to retain information stored therein. Operation of the storage device(s) of volatile storage 164 may be controlled by storage controller 165, which may receive commands from processor component 150 and/or other components of processing device 105 to store and/or retrieve information therein, and may convert those commands between the bus protocols and/or timings by which they are received and other bus protocols and/or timings by which the storage device(s) of volatile storage 164 are coupled to the storage controller 165. By way of example, the one or more storage devices of volatile storage 164 may be made up of dynamic random access memory (DRAM) devices coupled to storage controller 165 via an interface, for instance, in which row and column addresses, along with byte enable signals, are employed to select storage locations, while the commands received by storage controller 165 may be conveyed thereto along one or more pairs of digital serial transmission lines.

Non-volatile storage 162 may be made up of one or more storage devices that are non-volatile inasmuch as they are able to retain information stored therein without the continuous provision of electric power. Operation of storage device(s) of non-volatile storage 162 may be controlled by storage controller 165 (for example, a different storage controller than used to operate volatile storage 164), which may receive commands from processor component 150 and/or other components of processing device 105 to store and/or retrieve information therein, and may convert those commands between the bus protocols and/or timings by which they are received and other bus protocols and/or timings by which the storage device(s) of non-volatile storage 162 are coupled to storage controller 165. By way of example, one or more storage devices of non-volatile storage 162 may be made up of ferromagnetic disk-based drives (hard drives) operably coupled to storage controller 165 via a digital serial interface, for instance, in which portions of the storage space within each such storage device are addressed by reference to tracks and sectors. In contrast, commands received by storage controller 165 may be conveyed thereto along one or more pairs of digital serial transmission lines conveying read and write commands in which those same portions of the storage space within each such storage device are addressed in an entirely different manner.

Processor component 150 may include at least one processor core 170 to execute instructions of an executable routine in at least one thread of execution. However, processor component 150 may incorporate more than one of processor cores 170 and/or may employ other processing architecture techniques to support multiple threads of execution by which the instructions of more than one executable routine may be executed in parallel. Cache(s) 172 may include a multilayer set of caches that may include separate first level (L1) caches for each processor core 170 and/or a larger second level (L2) cache for multiple ones of processor cores 170.

In some embodiments in which processing device 105 includes display 138 and/or graphics controller 176, one or more cores 170 may, as a result of executing the executable instructions of one or more routines, operate controls 125 and/or the display 138 to provide a user interface and/or to perform other graphics-related functions. Graphics controller 176 may include a graphics processor core (for instance, a graphics processing unit (GPU)) and/or component (not shown) to perform graphics-related operations, including and not limited to, decompressing and presenting a motion video, rendering a 2D image of one or more objects of a three-dimensional (3D) model, etc.

Non-volatile storage 162 may store data 130, including executable instructions 132. In the aforementioned exchanges of data 130 between processing device 105 and server 110, processing device 105 may maintain a copy of data 130, for instance, for longer term storage within non-volatile storage 162. Volatile storage 164 may store encrypted data 134 and/or metadata 136. Encrypted data 134 may be made up of at least a portion of data 130 stored within volatile storage 164 in encrypted and/or compressed form according to some embodiments described herein. Executable instructions 132 may make up one or more executable routines such as an operating system (OS), device drivers and/or one or more application routines to be executed by one or more processor cores 170 of processor component 150. Other portions of data 130 may include data values that are employed by one or more processor cores 170 as inputs to performing various tasks that one or more processor cores 170 are caused to perform by execution of executable instructions 132.

As part of performing the executable instructions 132, one or more processor cores 170 may retrieve portions of executable instructions 132 and store those portions within volatile storage 164 in a more readily executable form in which addresses are derived, indirect references are resolved and/or links are more fully defined among those portions in the process often referred to as loading. As familiar to those skilled in the art, such loading may occur under the control of a loading routine and/or a page management routine of an OS that may be among executable instructions 132. As portions of data 130 (including portions of executable instructions 132) are so exchanged between non-volatile storage 162 and volatile storage 164, security subsystem 174 may convert those portions of data 130 between what may be their original uncompressed and unencrypted form as stored within non-volatile storage 162, and a form that is at least encrypted and that may be stored within volatile storage 164 as encrypted data 134 accompanied by metadata 136.

Security subsystem 174 may include hardware logic configured or otherwise controlled by security microcode 178 to implement the logic to perform such conversions during normal operation of processing device 105. Security microcode 178 may include indications of connections to be made between logic circuits within the security subsystem 174 to form such logic. Alternatively or additionally, security microcode 178 may include executable instructions that form such logic when so executed. Either security subsystem 174 may execute such instructions of the security microcode 178, or security subsystem 174 may be controlled by at least one processor core 170 that executes such instructions. Security subsystem 174 and/or at least one processor core 170 may be provided with access to security microcode 178 during initialization of the processing device 105, including initialization of the processor component 150. Further, security subsystem 174 may include one or more of the embodiments described herein for unified hardware acceleration of hash functions.

Security credentials 180 may include one or more values employed by security subsystem 174 as inputs to its performance of encryption of data 130 and/or of decryption of encrypted data 134 as part of performing conversions there between during normal operation of processing device 105. More specifically, security credentials 180 may include any of a variety of types of security credentials, including and not limited to public and/or private keys, seeds for generating random numbers, instructions to generate random numbers, certificates, signatures, ciphers, and/or the like. Security subsystem 174 may be provided with access to security credentials 180 during initialization of the processing device 105.

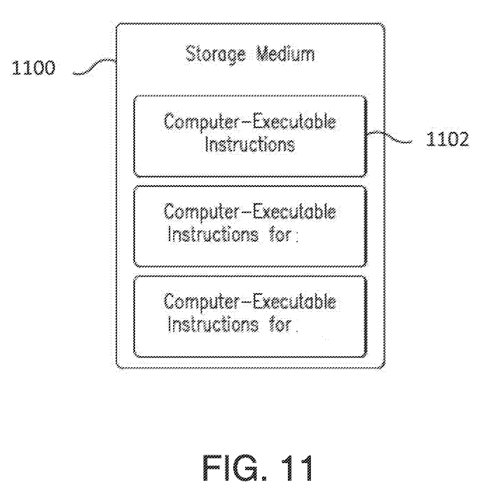

FIG. 11 illustrates an example of a storage medium 1100. Storage medium 1100 may comprise an article of manufacture. In some examples, storage medium 1100 may include any non-transitory computer readable medium or machine readable medium, such as an optical, magnetic or semiconductor storage. Storage medium 1100 may store various types of computer executable instructions, such as instructions 1102, which may correspond to any embodiment described herein, or to implement algorithms of energy delivery. Examples of a computer readable or machine readable storage medium may include any tangible media capable of storing electronic data, including volatile memory or non-volatile memory, removable or non-removable memory, erasable or non-erasable memory, writeable or re-writeable memory, and so forth. Examples of computer executable instructions may include any suitable type of code, such as source code, compiled code, interpreted code, executable code, static code, dynamic code, object-oriented code, visual code, and the like. The examples are not limited in this context.

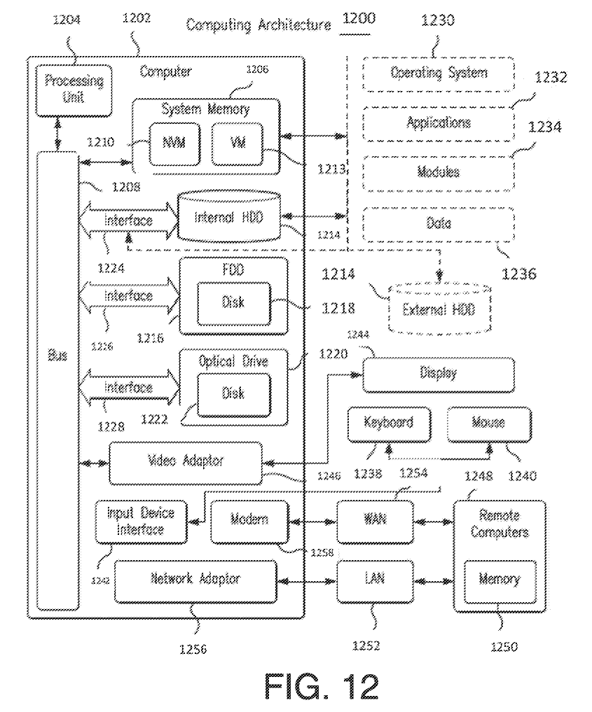

FIG. 12 illustrates an embodiment of an exemplary computing architecture 1200 suitable for implementing various embodiments as previously described. In one embodiment, the computing architecture 1200 may comprise or be implemented as part of an electronic device. Examples of an electronic device may include those described herein, such as electroporation delivery system 905 of FIG. 9 and processing device 105 of FIG. 10. The embodiments are not limited in this context.

As used in this application, the terms “system” and “component” are intended to refer to a computer-related entity, either hardware, a combination of hardware and software, software, or software in execution, examples of which are provided by the exemplary computing architecture 1200. For example, a component can be, but is not limited to being, a process running on a processor, a processor, a hard disk drive, multiple storage drives (of optical and/or magnetic storage medium), an object, an executable, a thread of execution, a program, and/or a computer. By way of illustration, both an application running on a server and the server can be a component. One or more components can reside within a process and/or thread of execution, and a component can be localized on one computer and/or distributed between two or more computers. Further, components may be communicatively coupled to each other by various types of communications media to coordinate operations. The coordination may involve the uni-directional or bi-directional exchange of information. For instance, the components may communicate information in the form of signals communicated over the communications media. The information can be implemented as signals allocated to various signal lines. In such allocations, each message is a signal. Further embodiments, however, may alternatively employ data messages. Such data messages may be sent across various connections. Exemplary connections include parallel interfaces, serial interfaces, and bus interfaces.

The computing architecture 1200 includes various common computing elements, such as one or more processors, multi-core processors, co-processors, memory units, chipsets, controllers, peripherals, interfaces, oscillators, timing devices, video cards, audio cards, multimedia input/output (I/O) components, power supplies, and so forth. The embodiments, however, are not limited to implementation by the computing architecture 1200.

As shown in FIG. 12, the computing architecture 1200 comprises a processing unit 1204, a system memory 1206 and a system bus 1208. The processing unit 1204 can be any of various commercially available processors, including without limitation an AMD® Athlon®, Duron® and Opteron® processors; ARM® application, embedded and secure processors; IBM® and Motorola® DragonBall® and PowerPC® processors; IBM and Sony® Cell processors; Intel® Celeron®, Core (2) Duo®, Itanium®, Pentium®, Xeon®, and XScale® processors; and similar processors. Dual microprocessors, multi-core processors, and other multi-processor architectures may also be employed as the processing unit 1204. For example, the unified hardware acceleration for hash functions described herein may be performed by processing unit 1204 in some embodiments.

The system bus 1208 provides an interface for system components including, but not limited to, the system memory 1206 to the processing unit 1204. The system bus 1208 can be any of several types of bus structure that may further interconnect to a memory bus (with or without a memory controller), a peripheral bus, and a local bus using any of a variety of commercially available bus architectures. Interface adapters may connect to the system bus 1208 via a slot architecture. Example slot architectures may include without limitation Accelerated Graphics Port (AGP), Card Bus, (Extended) Industry Standard Architecture ((E)ISA), Micro Channel Architecture (MCA), NuBus, Peripheral Component Interconnect (Extended) (PCI(X)), PCI Express, Personal Computer Memory Card International Association (PCMCIA), and the like.

The computing architecture 1200 may comprise or implement various articles of manufacture. An article of manufacture may comprise a computer-readable storage medium to store logic. Examples of a computer-readable storage medium may include any tangible media capable of storing electronic data, including volatile memory or non-volatile memory, removable or non-removable memory, erasable or non-erasable memory, writeable or re-writeable memory, and so forth. Examples of logic may include executable computer program instructions implemented using any suitable type of code, such as source code, compiled code, interpreted code, executable code, static code, dynamic code, object-oriented code, visual code, and the like. Embodiments may also be at least partly implemented as instructions contained in or on a non-transitory computer-readable medium, which may be read and executed by one or more processors to enable performance of the operations described herein.

The system memory 1206 may include various types of computer-readable storage media in the form of one or more higher speed memory units, such as read-only memory (ROM), random-access memory (RAM), dynamic RAM (DRAM), Double-Data-Rate DRAM (DDRAM), synchronous DRAM (SDRAM), static RAM (SRAM), programmable ROM (PROM), erasable programmable ROM (EPROM), electrically erasable programmable ROM (EEPROM), flash memory, polymer memory such as ferroelectric polymer memory, ovonic memory, phase change or ferroelectric memory, silicon-oxide-nitride-oxide-silicon (SONOS) memory, magnetic or optical cards, an array of devices such as Redundant Array of Independent Disks (RAID) drives, solid state memory devices (e.g., USB memory, solid state drives (SSD) and any other type of storage media suitable for storing information. In the illustrated embodiment shown in FIG. 12, the system memory 1006 can include non-volatile memory 1210 and/or volatile memory 1213. A basic input/output system (BIOS) can be stored in the non-volatile memory 1210.

The computer 1202 may include various types of computer-readable storage media in the form of one or more lower speed memory units, including an internal (or external) hard disk drive (HDD) 1214, a magnetic floppy disk drive (FDD) 1216 to read from or write to a removable magnetic disk 1218, and an optical disk drive 1220 to read from or write to a removable optical disk 1222 (e.g., a CD-ROM, DVD, or Blu-ray). The HDD 1214, FDD 1216 and optical disk drive 1220 can be connected to the system bus 1208 by a HDD interface 1224, an FDD interface 1226 and an optical drive interface 1228, respectively. The HDD interface 1224 for external drive implementations can include at least one or both of Universal Serial Bus (USB) and IEEE 1394 interface technologies.

The drives and associated computer-readable media provide volatile and/or nonvolatile storage of data, data structures, computer-executable instructions, and so forth. For example, a number of program modules can be stored in the drives and memory 1210, 1213, including an operating system 1230, one or more application programs 1232, other program modules 1234, and program data 1236. In one embodiment, the one or more application programs 1232, other program modules 1234, and program data 1236 can include, for example, the various applications and/or components to implement the disclosed embodiments.

A user can enter commands and information into the computer 1202 through one or more wire/wireless input devices, for example, a keyboard 1238 and a pointing device, such as a mouse 1240. Other input devices may include microphones, infra-red (IR) remote controls, radio-frequency (RF) remote controls, game pads, stylus pens, card readers, dongles, finger print readers, gloves, graphics tablets, joysticks, keyboards, retina readers, touch screens (e.g., capacitive, resistive, etc.), trackballs, trackpads, sensors, styluses, and the like. These and other input devices are often connected to the processing unit 1204 through an input device interface 1242 that is coupled to the system bus 1208, but can be connected by other interfaces such as a parallel port, IEEE 1394 serial port, a game port, a USB port, an IR interface, and so forth.

A display 1244 is also connected to the system bus 1208 via an interface, such as a video adaptor 1246. The display 1244 may be internal or external to the computer 1202. In addition to the display 1244, a computer typically includes other peripheral output devices, such as speakers, printers, and so forth.

The computer 1202 may operate in a networked environment using logical connections via wire and/or wireless communications to one or more remote computers, such as a remote computer 1248. The remote computer 1248 can be a workstation, a server computer, a router, a personal computer, portable computer, microprocessor-based entertainment appliance, a peer device or other common network node, and typically includes many or all of the elements described relative to the computer 1202, although, for purposes of brevity, only a memory/storage device 1250 is illustrated. The logical connections depicted include wire/wireless connectivity to a local area network (LAN) 1252 and/or larger networks, for example, a wide area network (WAN) 1254. Such LAN and WAN networking environments are commonplace in offices and companies, and facilitate enterprise-wide computer networks, such as intranets, all of which may connect to a global communications network, for example, the Internet.

When used in a LAN networking environment, the computer 1202 is connected to the LAN 1252 through a wire and/or wireless communication network interface or adaptor 1256. The adaptor 1256 can facilitate wire and/or wireless communications to the LAN 1252, which may also include a wireless access point disposed thereon for communicating with the wireless functionality of the adaptor 1256.

When used in a WAN networking environment, the computer 1202 can include a modem 1258, or is connected to a communications server on the WAN 1254, or has other means for establishing communications over the WAN 1254, such as by way of the Internet. The modem 1258, which can be internal or external and a wire and/or wireless device, connects to the system bus 1208 via the input device interface 1242. In a networked environment, program modules depicted relative to the computer 1202, or portions thereof, can be stored in the remote memory/storage device 1250. It will be appreciated that the network connections shown are exemplary and other means of establishing a communications link between the computers can be used.

The computer 1202 is operable to communicate with wire and wireless devices or entities using the IEEE 802 family of standards, such as wireless devices operatively disposed in wireless communication (e.g., IEEE 802.11 over-the-air modulation techniques). This includes at least Wi-Fi (or Wireless Fidelity), WiMax, and Bluetooth™ wireless technologies, among others. Thus, the communication can be a predefined structure as with a conventional network or simply an ad hoc communication between at least two devices. Wi-Fi networks use radio technologies called IEEE 802.11x (a, b, g, n, etc.) to provide secure, reliable, fast wireless connectivity. A Wi-Fi network can be used to connect computers to each other, to the Internet, and to wire networks (which use IEEE 802.3-related media and functions).

One or more aspects of at least one embodiment described herein may be implemented by representative instructions stored on a machine-readable medium which represents various logic within the processor, which when read by a machine causes the machine to fabricate logic to perform the techniques described herein. Such representations, known as “IP cores” may be stored on a tangible, machine readable medium and supplied to various customers or manufacturing facilities to load into the fabrication machines that actually make the logic or processor. Some embodiments may be implemented, for example, using a machine-readable medium or article which may store an instruction or a set of instructions that, if executed by a machine, may cause the machine to perform a method and/or operations in accordance with the embodiments. Such a machine may include, for example, any suitable processing platform, computing platform, computing device, processing device, computing system, processing system, computer, processor, or the like, and may be implemented using any suitable combination of hardware and/or software. The machine-readable medium or article may include, for example, any suitable type of memory unit, memory device, memory article, memory medium, storage device, storage article, storage medium and/or storage unit, for example, memory, removable or non-removable media, erasable or non-erasable media, writeable or re-writeable media, digital or analog media, hard disk, floppy disk, Compact Disk Read Only Memory (CD-ROM), Compact Disk Recordable (CD-R), Compact Disk Rewriteable (CD-RW), optical disk, magnetic media, magneto-optical media, removable memory cards or disks, various types of Digital Versatile Disk (DVD), a tape, a cassette, or the like. The instructions may include any suitable type of code, such as source code, compiled code, interpreted code, executable code, static code, dynamic code, encrypted code, and the like, implemented using any suitable high-level, low-level, object-oriented, visual, compiled and/or interpreted programming language.

Numerous specific details have been set forth herein to provide a thorough understanding of the embodiments. It will be understood by those skilled in the art, however, that the embodiments may be practiced without these specific details. In other instances, well-known operations, components, and circuits have not been described in detail so as not to obscure the embodiments. It can be appreciated that the specific structural and functional details disclosed herein may be representative and do not necessarily limit the scope of the embodiments.

Some embodiments may be described using the expression “coupled” and “connected” along with their derivatives. These terms are not intended as synonyms for each other. For example, some embodiments may be described using the terms “connected” and/or “coupled” to indicate that two or more elements are in direct physical or electrical contact with each other. The term “coupled,” however, may also mean that two or more elements are not in direct contact with each other, but yet still co-operate or interact with each other.

Unless specifically stated otherwise, it may be appreciated that terms such as “processing,” “computing,” “calculating,” “determining,” or the like, refer to the action and/or processes of a computer or computing system, or similar electronic computing device, that manipulates and/or transforms data represented as physical quantities (e.g., electronic) within the computing system's registers and/or memories into other data similarly represented as physical quantities within the computing system's memories, registers or other such information storage, transmission or display devices. The embodiments are not limited in this context.

It should be noted that the methods described herein do not have to be executed in the order described, or in any particular order. Moreover, various activities described with respect to the methods identified herein can be executed in serial or parallel fashion.

Although specific embodiments have been illustrated and described herein, it should be appreciated that any arrangement calculated to achieve the same purpose may be substituted for the specific embodiments shown. This disclosure is intended to cover any and all adaptations or variations of various embodiments. It is to be understood that the above description has been made in an illustrative fashion, and not a restrictive one. Combinations of the above embodiments, and other embodiments not specifically described herein will be apparent to those of skill in the art upon reviewing the above description. Thus, the scope of various embodiments includes any other applications in which the above compositions, structures, and methods are used.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

What is claimed is:

1. A system for regulating glucose levels, comprising:
a catheter having an expandable or inflatable portion;
one or more electrodes disposed on the expandable or inflatable portion of the catheter, wherein the one or more electrodes are configured to deliver energy to a patient's gastrointestinal tract; and
a drug delivery mechanism for delivering a drug therapy subsequent to energy delivery by the one or more electrodes.

2. The system according to claim 1, wherein the drug delivery mechanism includes a drug-coated balloon, one or more microneedles, an implantable device, or a hydrogel, or combinations thereof.

3. The system according to claim 1, wherein energy is deliverable by electroporation.

4. The system according to claim 1, wherein the drug therapy includes a growth inhibitor, cell cycle regulatory proteins/molecules, cyclin-dependent kinases, cell cycle inhibitors, cell regeneration inhibitor agents, or simulants of growth inhibitors, or combinations thereof.

5. The system according to claim 4, wherein the growth inhibitors include extracellular proteins, growth receptors, growth factors, transcriptional factors, cell adhesion molecules, cell signaling molecules, cytokines and chemokines, sulfate proteoglycans, chondroitin sulfate proteoglycans, enzymes, arginase, 13-secretase, or urokinase-type and tissue-type plasminogen activators, or combinations thereof.

6. The system according to claim 5, wherein the extracellular proteins include laminin, fibronectin, tenascin, fibrinogen, or fibrin, or combinations thereof.

7. The system according to claim 5, wherein the growth inhibitors include growth receptors including tyrosine kinase receptors (e.g., TrkA, TrkB, and/or TrkC), common neurotrophic receptor (e.g., P75NTR), ErbB receptors, or fibroblast growth factor receptors, or combinations thereof.

8. The system according to claim 5, wherein the growth factors include transforming growth factor alpha (TGF-α), epidermal growth factor (EGF), transforming growth factor beta (TGF-β), insulin-like growth factor (IGF), colony-stimulating factor (CSF), fibroblast growth factor (FGF), trefoil factor (TFF), hepatocyte growth factor (HGF), Glucagon-like peptide (GLP-2), or growth hormone (GH), or combinations thereof.

9. The system according to claim 5, wherein the transcriptional factors include the Hedgehog family, Forkhead Box (FOX) factors, Homeobox (HOX) genes, ParaHox genes, GATA transcription factors, canonical Wnt/β-catenin signaling, EPH/Ephrins, BMP signaling, K-RAS, Notch pathway, or HNF or MATH1, or combinations thereof.

10. The system according to claim 5, wherein the cell adhesion molecules (CAM) include N-CAM, Ng-CAM/L1, or N-cadherin or L2-HWk-1, or combinations thereof;
wherein the cell signaling molecules include Ras, Phosphotidyl Inositol 3-kinase, Phospholipase c-gamma 1, mitogen activated phosphor kinase, protein kinase A, Jaks/STATs signaling molecules, or combinations thereof; and
wherein the kinase inhibitors include staurosporine, H 89, dihydrochloride, cAMPS-Rp, triethylammonium salt, KT 5720, wortmannin, LY294002, 1C486068, 187114, GDC-0941, Gefitinib, Erlotinib, Lapatinib, AZ623, K252a, KT-5555, Cyclotraxin-B, Lestaurtinib, Tofacitinib, Ruxolitinib, SB1518, CYT387, LY3009104, TG101348, WP-1034, PD173074, or SPRY4, or combinations thereof.

11. The system according to claim 5, wherein the cytokines and chemokines include transforming growth factor-α, epidermal growth factor, interleukin-1β, or interferon-γ, or combinations thereof.

12. The system according to claim 5, wherein the sulfate proteoglycans include keratin sulfate proteoglycans, and wherein the chondroitin sulfate proteoglycans include neurocan, brevican, versican, phosphacan, aggrecan, or NG2, or combinations thereof.

13. The system according to claim 5, wherein the enzymes include targeting enzymes including Arginase I, Chondroitinase ABC, 13-secretase BACE1, urokinase-type plasminogen activator, or tissue-type plasminogen activator, or combinations thereof;
wherein Arginase I includes an N-hydroxy-L-arginine, or 2(S)-amino-6-boronohexonic acid, or combinations thereof;
wherein 13-secretase includes N-Benzyloxycarbonyl-Val-Leu-leucinal, H-Glu-Val-Asn-Statine-Val-Ala-Glu-Phe-NH2, or H-Lys-Thr-Glu-Glu-Ile-Ser-Glu-Val-Asn-Stat-Val-Ala-Glu-Phe-OH, or combinations thereof; and
wherein the urokinase-type and tissue-type plasminogen activators include serpin E1, Tiplaxtinin, or plasminogen activator inhibitor-2, or combinations thereof.

14. The system according to claim 4, wherein the cell cycle regulatory proteins/molecules include Cyclin A, Cyclin D, Cyclin D, Cyclin E, or Cyclin B, or combinations thereof;
wherein the cyclin-dependent kinases include Cdk1, Cdk2, Cdk3, Cdk4, or Cdk6, or combinations thereof;
wherein the cell cycle inhibitors include p21, p27, or p57, or combinations thereof;
wherein the cell regeneration inhibitor agents include paclitaxel, dual phosphate and tensin homolog (PTEN), or SCOS3, or combinations thereof; and
wherein simulants of growth inhibitors include INK4a/ARF families including p16 and p14.

15. The system according to claim 1, wherein the one or more electrodes are configured to deliver an amount of energy sufficient to ablate tissue of the patient's gastrointestinal tract, and the drug therapy is deliverable in an amount sufficient to inhibit subsequent cell growth of the tissue.

16. A method for regulating glucose levels, comprising:
inserting a catheter into a patient's gastrointestinal tract;
positioning the catheter in a duodenum of the patient's gastrointestinal tract;
expanding or inflating a portion of the catheter in the duodenum, the expandable or inflatable portion of the catheter including one or more electrodes;
applying energy to the duodenum via the one or more electrodes to ablate tissue of the duodenum; and
delivering a drug therapy to the ablated tissue of the duodenum.

17. The method according to claim 16, wherein the drug therapy is delivered by a drug-coated balloon, one or more microneedles, an implantable device, or a hydrogel, or combinations thereof.

18. The method according to claim 16, wherein the application of the energy and delivery of the drug therapy alters how the patient's body regulates glucose levels.

19. The method according to claim 16, wherein the drug therapy includes a growth inhibitor, cell cycle regulatory proteins/molecules, cyclin-dependent kinases, cell cycle inhibitors, cell regeneration inhibitor agents, or simulants of growth inhibitors, or combinations thereof.

20. A method of treating diabetes, comprising:
applying electroporation energy to a duodenum of a patient to ablate tissue of the duodenum; and
delivering a drug therapy to the ablated tissue of the duodenum;
wherein the drug therapy includes a growth inhibitor, cell cycle regulatory proteins/molecules, cyclin-dependent kinases, cell cycle inhibitors, cell regeneration inhibitor agents, or simulants of growth inhibitors, or combinations thereof.
